(19) United States
(12) Patent Application Publication
(10) Pub. No.: US 2006/0292038 A1
(43) Pub. Date: Dec. 28, 2006

(54) Automated sample analyzer and cuvette

(76) Inventors: Henrik Lennart Johansson, Esbo (FI); Juhani Olavi Makuhen, Mustio (FI)

Correspondence Address: STEPHAN A. PENDORF, P.A., PENDORF & CUTLIFF, 5111 MEMORIAL HIGHWAY, TAMPA, FL, 33634, US

(21) Appl. No.: 10/545,391

(22) PCT Filed: Jan. 15, 2004
(86) PCT No.: PCT/EP04/00415

§ 371 Date: May 9, 2006

(30) Foreign Application Priority Data
Feb. 14, 2003 (GB) 0303453.5

(51) Int. Cl. C12M 1/34; G01N 21/00

(52) U.S. Cl. 422/82.05; 435/287.2

(57) ABSTRACT

An automated sample analyzer (10) has a sample store (20) and a sample dispenser (90) which is controlled by a controller (170). Samples of a substance to be analyzed are dispensed into a cuvette (60), along with a reagent from a reagent storage compartment (40), after which incubation occurs in an incubator (70). The incubated analyte is then passed to a sample analysis module (140). This has a photometric analysis arrangement (150) and a twin photon excitation (TPX) analysis arrangement (160). The incubated analyte is analyzed using one or other of the arrangements (150, 160). By parallel processing of samples in the analysers (150), (160), a rapid throughput of samples is possible but with the possibility of both photometric and TPX analysis. The cuvette (60) is of a tapered design which is particularly suitable for samples to be analysed by either arrangement (150, 160).

### FIELD OF THE INVENTION

This invention relates to an analyzer for automated analysis of biological and/or chemical samples. The invention also relates to a cuvette, particularly but not exclusively suitable for such an analyzer.

### BACKGROUND TO THE INVENTION

Many systems for performing routine, clinical or immuno-chemical analysis are currently available. Automation of the process at least in respect of chemical analysis for routine chemistry is relatively well established. For example, the Konelab™ analyzers produced by Thermo Clinical Labsystems of Finland permit automated analysis of various clinical chemicals, proteins, electrolytes, narcotics and the like, with (in the larger instruments) up to 600-800 samples capable of being analyzed each hour. The Konelab™ device employs a multicell cuvette, which receives a sample to be analyzed and a reagent from separate storage regions. The sample and reagent are mixed in the cuvette, incubated, and chemical analysis is then carried out using photometric techniques that will be familiar to those skilled in the art.

Automated immunoassay analyzers have also become increasingly available over recent years. However, automation of the heterogeneous immunoassay procedure is laborious since there are a large number of steps in the procedure. Typically, a sample is mixed with a reagent and a solid support having a bound antigen or antibody. The sample is incubated in such a way that the corresponding antigen or antibody in the reagent can be bound to the antigen or antibody on the solid support. The support is then thoroughly washed (separated) and a label, typically flourescent, chemiluminescent or similar, is detected in an appropriate manner. The analyte of interest is then quantified from the detected label.

The current requirements for traditional clinical chemistry as described above are thus significantly different to the current requirements for immunochemistry. As such, it is usual to employ separate devices for the separate analyses, since there is little in common between the components and procedures for each device.

### SUMMARY OF THE INVENTION

According to a first aspect of the present invention, there is provided an automated sample analyzer comprising: a sample repository arranged to hold a plurality of samples to be analyzed; a reagent repository arranged to hold a plurality of reagents to be mixed with the samples; an analyte preparation station arranged to receive a quantity of sample from the sample repository, to receive a quantity of at least one reagent from the reagent repository, and to mix these together to form an analyte; a photometric analysis assembly arranged to carry out a photometric analysis of a material; a twin photon excitation (TPX) assembly arranged to carry out twin photon excitation (TPX) analysis of a material; and a controller to divert the analyte from the analyte preparation station to the photometric analysis assembly or the TPX analysis assembly in dependence upon the desired type of analysis of the analyte.

The present invention provides, in general terms, a combined automated sample analyzer capable of handling both traditional (clinical) chemical assays and also immunoassays. The analyzer uses separate devices for measuring/detecting the samples, depending upon the type of analysis required. For routine chemical analysis, a photometer is employed whereas for immunochemical analysis, so-called two photon excitation (TPX) measurements are made. Using this technique, it is possible to dispense with the washing or separation steps and to take TPX measurements directly from the reaction mixture, measured from the surface of the supporting micro-particle. All liquid handling steps, reagent mixing and, where necessary, incubation can thus be handled by common parts whether routine chemical or immunoassay analysis is to be carried out. The only requirement is that one or other of a photometer or a TPX measuring device is selected in accordance with the type of assay to be analyzed. There is thus a significant reduction in cost and complexity of an analyzer relative to the prior art, with increased flexibility in terms of types of material that can be analyzed.

A further benefit of the present invention is that measurements using TPX can be carried out using extremely low volumes of fluid. This in turn enables a considerable saving in reagent costs to be realised.

The present invention also relates to a new cuvette. The cuvette is a small sample holder or reaction vessel which, for different applications, may be single cell or multi-cell. In order to allow optical access to the analyte within the or each cell, the walls of the cell are translucent so as to permit optical measurements. One such prior art cuvette is disclosed in U.S. Pat. No. 4,690,900. It will be seen that each cell within the interlocking unitary cuvettes is generally rectangular in section with a flat base to each. The minimum fill volume of such a cell is about 100 82 l and is optimised for prior art analyzer.

The present invention accordingly provides a cuvette for use in an automated sample analyzer, the cuvette comprising a chamber having chamber walls which define a chamber opening and first and second chamber volumes, wherein the first volume is proximal the opening, the second volume is remote from the opening, and wherein the first volume has a transverse sectional area larger than the transverse sectional area of the second volume.

Such a cuvette provides several advantages, such as the ability to use the smaller second volume for TPX analysis (which reduces reagent wastage), whilst still maintaining a suitable volume (by filling the first and the second volumes) for photometric analysis. The second volume in particular may be curved at its base so as to permit better stirring of sample and reagent. There may also be a heat buffer such as an air gap between adjacent chambers where more than one chamber is provided in the cuvette.

The cuvette of the present invention provides particular benefits when used with the combined photometric/TPX analyzer. However, it is to be appreciated that the cuvette of the present invention is not restricted to such uses. The advantages of the cuvette and hence its range of applications will become apparent upon review of the following description of a preferred embodiment.

The invention also extends to a method of automated sample analysis comprising the steps of: (a) storing at least one sample to be analyzed; (b) storing, separately, at least one reagent to be mixed with the sample(s); (c) receiving a quantity of a sample and a quantity of the, or at least one reagent; (d) mixing the quantities of sample and reagent(s) to form an analyte; and (e) directing the analyte to a selected one of a photometric analysis assembly and a twin photon excitation (TPX) analysis assembly in dependence upon the desired type of analysis of the analyte.

### BRIEF DESCRIPTION OF THE DRAWINGS

The invention may be put into practice in a number of ways, and one specific embodiment will now be described by way of example only, with reference to the accompanying drawings in which:

FIG. 1 shows a highly schematic plan view of an automated sample analyser embodying an aspect of the present invention and including TPX and photometric analysis arrangements;

FIG. 2a shows, in more detail, a schematic of the photometric analysis arrangement of FIG. 1;

FIG. 2b shows, in more detail, a schematic of the TPX analysis arrangement of FIG. 1;

FIG. 3a shows a side view of a cuvette embodying a further aspect of the present invention; and

FIG. 3b shows a top view of the cuvette of FIG. 3a.

### DETAILED DESCRIPTION OF THE PREFERRED EMBODIMENTS

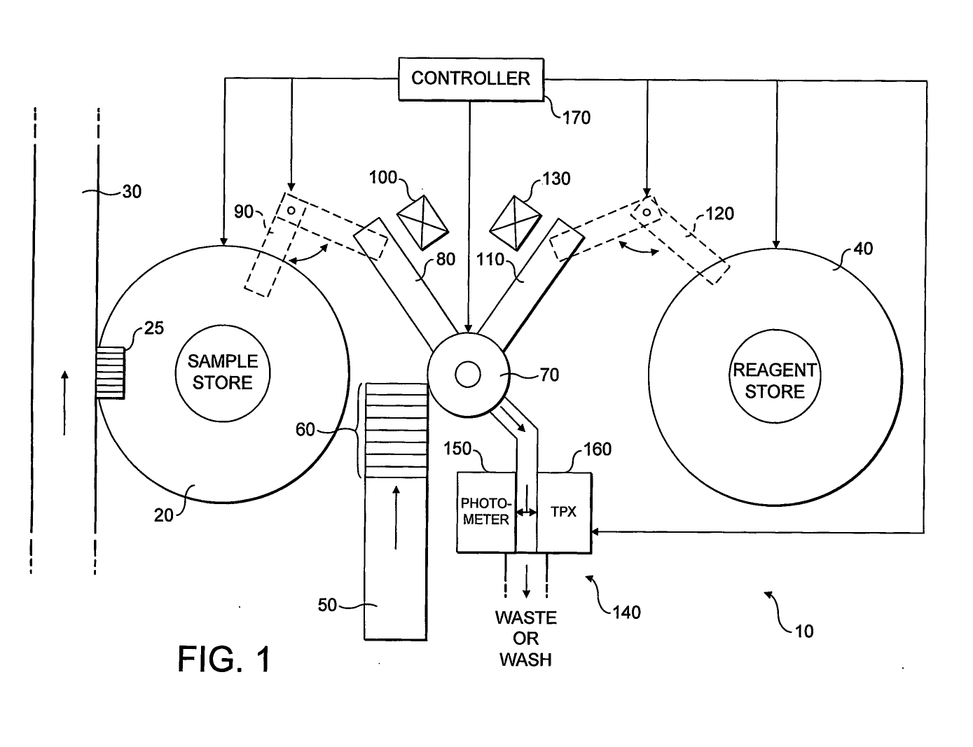

Referring first to FIG. 1, a highly schematic top view of an automated analyzer embodying an aspect of the present invention is shown. The analyzer is indicated generally at reference 10 in FIG. 1.

Samples to be analyzed, such as blood or urine samples, as well as control materials and analytical standard solutions for system calibration, are held during operation of the analyzer 10 in a sample storage compartment 20. The sample storage compartment 20 is, in the preferred embodiment, a carousel capable of holding a plurality of samples and other solutions.

Typically, samples and the like are loaded into the sample storage compartment 20 in cups or tubes (not shown). These in turn are held in sample racks (again not shown) which are introduced into the sample storage compartment 20 through an entry/exit 25. The sample racks can be introduced either manually or, for example, from a sample transport line 30 as is known in the art.

Once each sample rack is loaded into the entry/exit 25 of the sample storage compartment 20, it is automatically transported from there into the carousel. The sample storage compartment 20 typically holds several racks, each containing a quantity of samples, and random access to any of the samples within the sample storage compartment 20 is subsequently possible. The sample racks are identified by bar codes and, preferably, the cups or tubes which hold the samples themselves may also be labelled with individual bar codes so that individual samples and racks can each be identified by a bar code reader.

Once the samples have been analyzed in a manner to be described below, they are ejected from the sample storage compartment 20, again through the entry/exit 25, for waste disposal.

The analyzer 10 also includes a reagent storage compartment 40. The reagent storage compartment 40 holds reagents for the different types of analysis to be carried out by the analyzer 10, potentially in different-sized containers. As with the sample storage compartment 20, any reagents within the reagent storage compartment 40 can be preferably labelled with a bar code label to allow identification and random access for subsequent sample analysis.

Both the sample storage compartment 20 and the reagent storage compartment 40 are thermally isolated from the rest of the analyzer and cooled so as to permit optimal storage conditions for the samples and reagents respectively.

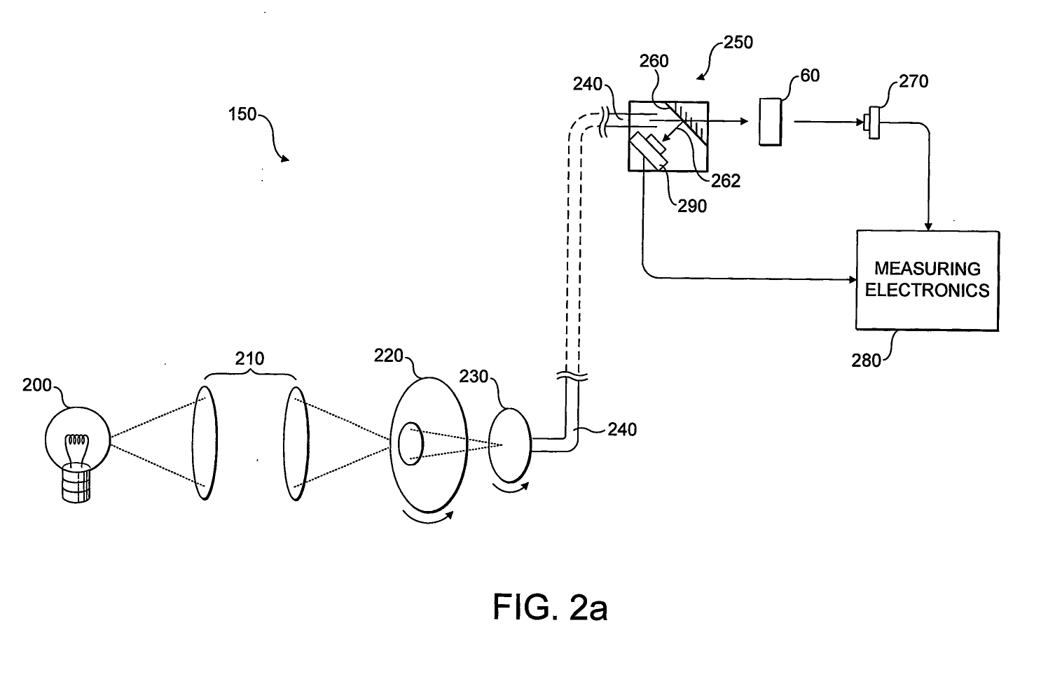

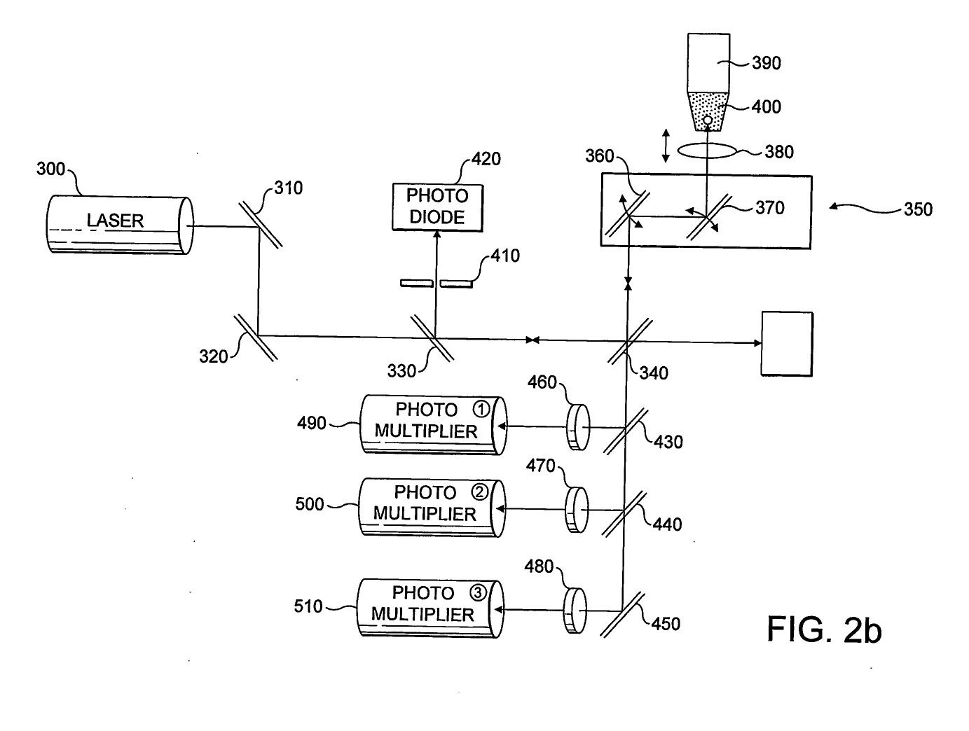

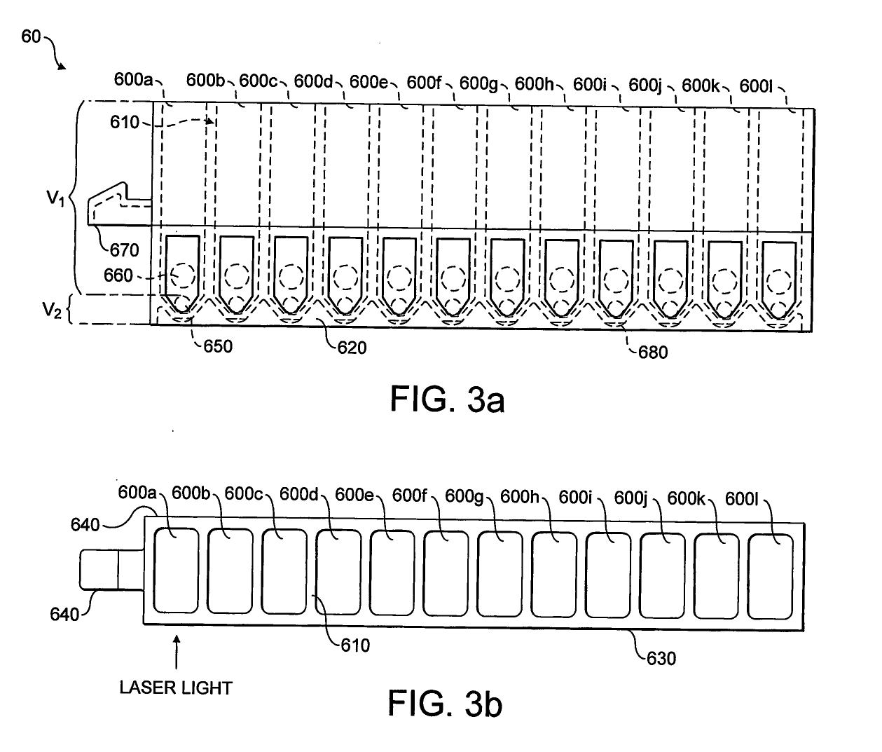

In the arrangement of FIG. 1, analysis of samples is carried out in a cuvette. The details of the preferred cuvette for the analyzer 10 of FIG. 1 will be set out in connection with FIGS. 2 and 3 below. In general terms, however, cuvettes are loaded into the analyzer 10 via a cuvette loader 50. The cuvette loader 50 allows storage of rows of empty cuvettes 60 (either disposal or washable) which are loadable sequentially into an incubator 70.

The incubator 70 has several entrances. A first entrance allows loading of a cuvette as described above. A second entrance to the incubator 70 is from a sample dispensing station 80. The sample dispensing station 80 acts as a transit point between the sample storage compartment 20 and the incubator 70. In particular, samples are extracted from the sample storage compartment 20 using a sample dispenser 90 and added to a cuvette which has been loaded into the sample dispensing station 80 from the incubator 70. The sample dispensing station 80 is preferably heated using one or more resistors so as to maintain a temperature therein. A sample stirrer 100 is also provided to stir the sample once it has been placed into the cuvette at the sample dispensing station 80. The details of this procedure will be described in connection with the analysis procedure below.

The incubator 70 also has an entrance/exit for a reagent dispensing station 110. In analogous fashion to the sample dispensing station 80, the reagent dispensing station 110 acts as an intermediate location between the reagent storage compartment 40 and the incubator 70. The reagent dispensing station 110 takes cuvettes from the incubator 70 which are filled by a reagent dispenser 120. The reagent dispensing station 110 may likewise he heatable using one or more resistors. A reagent stirrer 130 is provided adjacent to the reagent dispensing station 110 to stir reagents added to the cuvettes there.

The incubator itself is a thermostatic aluminium block, placed inside an isolated chamber. The aluminium blocks holds the cuvettes as they pass through the system, warms them to a desired temperature and maintains a stable temperature during incubation of a reaction mixture, that is, a mixture of a sample from the sample storage compartment 20 and one or more reagents from the reagent storage compartment 40. The incubator also acts as a cuvette exchange centre (in effect, a turntable) to direct the cuvettes onto the sample dispensing station, back off that sample dispensing station, onto the reagent dispensing station, back off the reagent dispensing station, and, ultimately, out of the incubator for subsequent analysis of the incubated reaction mixture or analyte.

The final part of the analyzer 10 is a sample analysis module 140. This is located downstream of the incubator 70 and receives incubated analyte. The sample analysis module 140 comprises a photometric analysis arrangement 150, and a twin photon excitation (TPX) analysis arrangement 160. A schematic diagram of the components within the photometric analysis arrangement 150 is shown in FIG. 2a. A similar schematic diagram of the components or the TPX analysis arrangement 160 is shown in FIG. 2b. Further details of preferred embodiments of these analyzers will be set out below. In general terms, however photometer operate on the basis of Beer's law, in which the concentration of light absorbing species in a liquid is proportioned to the absorbence of electromagnetic radiation (light) for a given path length through the sample and at a particular wavelength. This allows measurements by directing electromagnetic radiation through a cell containing a fluid sample and then measuring the characteristics of the light transmitted through the cell. The light transmitted is normally compared to transmittance for liquids with a known concentration. From this data, a calibration curve can be established. This calibration curve enables the calculation of the actual concentration to be carried out.

Twin photon excitation, by contrast, is a technique used to analyze bioaffinity assays. The method is based upon the optical elimination of unwanted background signals outside an observation volume, thus enabling the observation of binding reaction without the need to carry out a separation or washing step.

Two photon excitation is caused only inside a three-dimensional diffraction limited focal volume of the focused laser light, leading to an extremely low effective volume. This observation volume depends on the optical parameters such as the wavelength of excitation light and the numerical aperture of the particular optical system.

The basis of the chemical principle is the use of coated biochemically active polystyrene micro-particles as solid phase carriers, and a reagent labelled with a fluorescent label. The label either competes with the analyte for binding sites on the particle, or binds to a second epitope of the analyte and forms a molecular sandwich, this depending upon the type of chemistry chosen.

Since the micro-particles concentrate the label on their surface, the signal from the surface can be orders of magnitude stronger than the signal from the unbound label within the reaction solution and no prior separation procedure will now be needed. The signal from the micro-particle surface indicates the degree of binding.

Depending upon the nature of the sample to be analyzed, the sample analysis module diverts the cuvette, containing analyte, into the photometric analysis arrangement 150 or the TPX analysis arrangement 160. Once analysis has taken place, the cuvettes are ejected from the sample analysis module 140 to waste or to be washed.

The analyzer 110 is under the control of a controller 170 which is typically a personal computer or other processor. The controller governs the collecting of samples and reagents from the sample and reagent storage compartments 20, 40 respectively (using the bar codes on the sample storage cups/tubes and reagent bottles), the loading of cuvettes from the cuvette loader 50 and the movement of the incubator 70 to control passage of cuvettes onto and off the sample and reagent dispensing stations 80, 110, the stirring of the sample/reagent, ejection from the incubator 70, diversion to one or other of the photometric or TPX analysis arrangements 150, 160 and so forth. The controller 170 may also gather the results of the analyses for database storage or visual display, for example.

Having described, in overview, the analyser 10 of FIG. 1, an example of the method steps taken in the preparation and analysis of a sample will now be described, with reference again to FIG. 1 and, subsequently, to FIGS. 2a and 2b. In general terms, the procedure for processing a sample is the same whether photometric or TPX analysis is to be carried out.

Firstly, a cuvette is pushed into an empty slot in the incubator 70 from the cuvette loader 50. The empty cuvette from the cuvette loader 50 is pre-incubated in the incubator 70 for a few minutes, after which it is passed to the reagent dispensing station 110. Once the cuvette is loaded onto the reagent dispensing station 110, the reagent storage compartment 40 rotates so that the appropriate reagent (which has been selected by a user either directly or through the use of a standard assay procedure in the controller 170) may be accessed by the reagent dispenser 120.

The reagent dispenser 120 is a dispensing robot connected to a syringe (not shown) and is capable of metering the uptake and dispensing of a reagent from the reagent storage compartment 40. To detect the surface of the reagent liquid (and thus ensure the correct volume of reagent is collected) the needle of the reagent dispenser 120 is equipped with a level sensing system (not shown). This level sensing system can also measure the amount of reagent left in the reagent bottle.

As will be explained in further detail below, the amount of reagent necessary is dependent, as a rule, upon the type of analysis that is to take place. Specifically, the amount of reagent necessary for TPX analysis is generally significantly less than the amount of reagent that is necessary for photometric analysis. Therefore, the controller 170 controls the amount of reagent that is collected by the reagent dispenser 120 from the reagent storage compartment 40 in dependence upon the intended analysis procedure.

The arm of the reagent dispenser 120 turns until it is correctly aligned with the appropriate reagent bottle in the reagent storage compartment 40 and then the needle of the reagent dispenser 120 moves down into the bottle until surface contact with reagent is made. When contact has been made, a predetermined volume of reagent is sucked up with the metering syringe. The arm of the reagent dispenser 120 then rotates until it is aligned with a first cell in the cuvette which has been loaded onto the reagent dispensing station 110. The reagent is then ejected from the syringe into that first cell and the needle is then drawn out of the cuvette.

Next, the reagent dispenser 120 rotates to a washing station, which is not shown in FIG. 1 for the sake of clarity. The dispensing needle which has previously dispensed the reagent into the cell of the cuvette is washed at that wash station to avoid cross-contamination with subsequent reagents.

In the meantime, the reagent stirrer 130 stirs the recently dispensed reagent. As will be understood by those skilled in the art, there are many different ways of stirring a liquid. In the present embodiment, a helical paddle is employed within the reagent stirrer 130 and this is of a dimension to allow it to be inserted within a cell of a cuvette. The reagent stirrer 130 includes a wash station to allow the mixing paddle to be washed after each use.

Once the needle within the reagent dispenser has been washed, the reagent dispenser 120 rotates again to access the reagent storage compartment 40 once more. Either the same reagent can be obtained, or alternatively the reagent storage compartment 40 can be rotated so as to allow access to a different reagent bottle. The reagent dispenser 120 then moves once more to dispense that next reagent into a second cell within the sample cuvette on the reagent dispensing station 110. This is also mixed whilst the needle of the reagent dispenser 120 is washed once more.

This procedure continues until all of the cells within the cuvette have had reagent dispensed into them, or, alternatively, until a sufficient number of cells have been filled to carry out all of the sample analyses requested.

At this point, the filled or partially-filled cuvette is moved from the reagent dispensing station 110 back into the incubator 70. From there, the reagent-filled cuvette is moved onto the sample-dispensing station 80. However, a measurement of the reagent prior to mixing with a sample may be carried out with the photometric or TPX analysis arrangement, to obtain calibration information, for example.

Once the cuvette with reagent in it has been moved onto the sample-dispensing station 80, the sample storage compartment 20 is rotated so that the appropriate sample cup or tube to be processed is aligned with the sample dispenser 90. As with the reagent dispenser 120, the sample dispenser 90 has an arm with a syringe on it, and that syringe has a needle which is equipped with a level-sensing system to determine the amount of sample which is collected, and likewise the amount of sample left in the sample cup or tube.

The arm of the sample dispenser 90 moves over the appropriate sample and the needle of the sample dispenser 90 moves down into the sample cup or tube until the surface of the sample liquid is detected. Once the surface has been located, the predetermined sample volume is aspirated by the sample syringe. That volume may again differ depending upon the intended analysis (TPX or photometric), and the controller may control the sample dispenser 90 appropriately. Next, the needle is extracted from the sample and the arm of the sample dispenser 90 moves until the tip thereof is aligned with a cell within the cuvette. The sample is discharged into the appropriate cell and the needle is then removed. The sample stirrer 100, which, like the reagent stirrer is preferably a helical paddle sized to fit into the cell of the cuvette, stirs the liquid (sample and reagent) in that cell. The needle of the sample dispenser 90 and the sample stirrer 100 are washed at respective washing stations which, again, are not shown in FIG. 1 for the sake of clarity.

Once all of the cells in the cuvette on the sample dispensing station 80 are filled, or at least, all of those cells with reagent in them have been filled, the cuvette is moved back off the sample dispensing station 80 and into the incubator 70 once more. Next, the cuvette may be moved, by rotation of the incubator 70 back out onto the reagent dispensing station 110 again, to allow addition of further reagents to the sample/reagent mixture. In that case, the procedure is as previously until the cuvette is back in the incubator 70 once more. At that stage, to complete the chemical reaction, it is usually necessary to incubate the sample/reagent mixture in the incubator 70. This takes place for a preset incubation time. Once incubation is complete, the cuvette is ejected from the incubator 70 towards the sample analysis module 140. The controller then governs whether the cuvette is moved into the photometric analysis arrangement 150 or the TPX analysis arrangement 160, in dependence upon the type of analysis to be carried out. It will be appreciated that, whilst it is possible to carry out photometric and TPX analysis on different cells in a single cuvette, it is generally more efficient to use different cuvettes for different analyses so that, for example, in a cuvette having twelve cells, all twelve are used for photometric analysis whilst a different (but preferably identical in appearance) cuvette is used for samples that are to be analyzed using the TPX analysis arrangement 160.

After measurements have been taken, the cuvette is ejected to waste or for washing whereupon the next cuvette can be used for analysis. Again, if both photometric and TPX analysis is to be carried out on either the same or different samples, it will be understood that parallel processing of these can take place—that is, it is not necessary for photometric analysis to be carried out serially with TPX analysis and separate cuvettes can be analyzed in the separate arrangements 150, 160 simultaneously. This allows a throughput of samples which is approaching that of separate analyzers, one with only photometric analysis apparatus and one with only TPX analysis apparatus, but in the apparatus of FIG. 1, using only a single sample store, reagent store, controller and dispensing stations etc.

FIGS. 2a and 2b show, in further detail but still in schematic form, the optical arrangements for the photometric analysis and TPX analysis arrangements 150, 160 respectively. Referring first to FIG. 2a, the photometric analysis arrangement 150 is shown in more detail. The arrangement 150 includes a light source 200 which is, in the preferred embodiment, a tungsten halogen lamp. Radiation from the tungsten halogen lamp is focused via a lens 210 onto a light guide 240 such as a fibre optic cable, only the entrance and exit of which is shown in FIG. 2a.

An interference filter 220 and light chopper 230 are interposed between the lens 210 and the entrance to the light guide 240. The interference filter 220 acts as a band pass filter to allow passage of radiation only of a certain bandwidth into the light guide. Typically, this bandwidth is 340 nm-880 nm. The interference filter 220 is preferably formed as a filter wheel carrying a plurality of filters having different pass bands. The desired pass band can then be chosen by rotating the filter wheel.

The filtered light passes through the light chopper 230 which modifies the radiation into modulated, chopped light. Speed of the chopper 230 is maintained constant by a controlling microprocessor (not shown).

The light guide 240 directs light exiting the chopper 230 to a measuring head indicated at reference numeral 250 in FIG. 2a. On entry into the measurement head, a beam splitter 260 divides the light beam into two separate beams, a reference beam 262 and a measurement beam 264.

The reference beam 262 is passed to a reference detector 290, where the intensity of the light is detected by a photodetector. The measuring beam is restricted by apertures (not shown) and then directed through a cuvette 60 which contains the analyte which is to be measured. From there, the light passes to a measurement detector 270 which generates an electrical signal proportional to the intensity of the radiation which has been transmitted through the liquid analyte in the cuvette 60. By moving the cuvette across the measurement beam, liquids in different cells within the cuvette 60 can be analysed sequentially.

The reference detector likewise generates an electrical signal proportional to the intensity of the reference beam. These signal outputs of the measurement and reference detectors 270, 290 are both passed to measuring electronics 280 which compares the outputs of these two detectors with values obtained from a non-absorbing liquid such as water. From this, the transmission of radiation through the cuvette 60 can be determined and thus the concentration in the analyte can be established.

FIG. 2b shows, again schematically, the details of the TPX analysis arrangement 160. The optical arrangement includes a passively Q-switched, diode-pumped, microchip Nd:YAG laser 300. This operates at a wavelength of 1064 nm and has a pulse duration less than 1 ns with a repetition rate of around 17 kHz. Light emanating from the laser 300 is reflected off first and second mirrors 310, 320. It then passes through a first dichroic mirror which acts as a back-scattering mirror 330. The laser light is then reflected at a second dichroic mirror 340 into a scanning arrangement 350. The scanning arrangement 350 comprises first and second movable scanning mirrors 360, 370 which allow the laser light to be directed in different directions.

The laser light is tightly focused through a microscope objective lens 380 which has a typical N.A. of 0.65 and into a cuvette 390 containing the analyte 400.

When the laser beam reaches the analyte 400, and hits a micro-particle within that analyte 400, the micro-particle itself will back-scatter the incident light, and also fluoresce from the surface of the micro-particle.

The back-scattered signal passes back through the scanning arrangement 350, is reflected off the second dichroic mirror 340 and is then reflected again by the back-scattering mirror 330. The back-scattering mirror 330 thus diverts back-scattered light from the analyte 400 through a pin hole 410 having a typical diameter aperture of around 80 μm. The back-scattered signal is thus focused through that pin hole 410 and is eventually detected by an InGaAs photodiode 420.

The back-scattered signal is used to control the fluorescence measurement procedure, that is, to ascertain when the laser beam is striking a micro-particle. It also allows determination of an appropriate time to carry out fluorescence measurement. This will be described in further detail below.

The two-photon excited fluorescence signal from the analyte is directed through the scanning mirrors 360 and 370 in the scanning arrangement 350 to the second dichroic mirror 340. Here, the fluorescent light passes through the second dichroic mirror 340 and is then separated into three different wavelength ranges using third, fourth and fifth dichroic mirrors 430, 440, 450, before being further filtered by band pass filters 460, 470, 480 with corresponding first, second and third photo-multipliers 490, 500, 510. The three photo-multipliers act as detectors.

The analyte contains a huge amount of micro-particles. One assay measurement involves the determination of the fluorescence of several micro-particles, from which values a mean value is calculated in accordance with an algorithm. A mean value, from several different micro-particles, is obtained so as to minimize the degree of statistical fluctuation. This mean value represents the final bioaffinity value.

The scanning arrangement 350 of the TPX analysis arrangement 160 allows rapid location of new particles for measurement. In particular, the first and second movable scanning mirrors 360, 370 are tiltable relative to one another to allow tilting of the incident laser beam by 2-3°. The confocal back-scattering signal is continually measured by the photodiode 420 during the scan, and when the back-scattering signal rises above a given threshold, the movement of the scanning mirrors 360, 370 is stopped and the fluorescence measurement commences.

The optical pressure of the laser beam traps the micro-particles centering it in the lateral direction and pushes it in the axial direction through the focus of the laser. The fluorescent signal is measured when the back-scattering signal exceeds the given threshold, which implies that the micro-particle is in focus. The measurement time for a single micro-particle is around 50-ms. When the micro-particle has left the focus (again, indicated by the magnitude of the back-scattering signal), xy scanning commences again by-moving the scanning mirrors 360, 370 until the next micro-particle is located.

The combined scanning and fluorescence measuring time allows between 5 and 10 micro-particles to be measured per second. Only a fraction of all of the micro-particles within the assay volume of the analyte 400 are actually measured.

The procedure continues until a predetermined amount of particles has been measured, or until a predetermined time period has elapsed. The data generated is then used to generate a bio-affinity value for the analyte 400. This value may be compared to a calibration curve produced using calibrators with known bio-affinity values. This allows the analyte concentration to be determined.

The apparatus 160 also contains a photodetector 345 which receives light via the dichroic mirror 340, directly from the laser 300. The photodetector 345 acts to trigger measurement by the analyser 160, when the light from the laser is received in it.

The type of cuvette which may be employed with the analyzer 10 of FIG. 1 is not critical to its operation. For example, the cuvette as described in U.S. Pat. No. 4,690,900 will permit adequate operation of the analyzer of FIG. 1. However, an improved cuvette, with features that are particularly beneficial to the operation of the analyzer of FIG. 1, has been developed, and this is shown in side and plan views, in FIGS. 3a and 3b respectively.

The cuvette 60 is formed of a translucent material such as glass or, more usually, a plastics material and comprises a plurality of cells 600 divided from adjacent cells by a party or dividing wall 610. In the example of FIGS. 3a and 3b, there are twelve cells 600a-6001 separated by eleven dividing walls 610. The cuvettes of FIGS. 3a and 3b are shown slightly larger than 1:1 scale.

In contrast to prior art cuvettes, the cells 600 of the cuvette 60 of FIG. 3a and FIG. 3b comprise a non-uniform section. In particular, the bottom of each cell is narrower than the top of each cell, as may best be seen in FIG. 3a. In that Figure, it will be seen that a first, relatively smaller volume V₂ is defined by a tapered region at the bottom of each cell, with a relatively larger volume V₁ defined above that tapered volume V₂. The larger volume V₁ is, in the preferred embodiment, of substantially uniform diameter.

The separation between adjacent cells is thus larger at the bottom of each cell than at the top—that is, the separate between the lower, smaller volumes V₂ is greater than the separation between the upper volumes V₁ of adjacent cells. In the preferred embodiment, the lower volumes V₂ are separated by an air gap 620. The benefit of this will be explained below.

Photometric analysis of samples, a relatively large volume of analyte is necessary, for example somewhere between 120 and 200 μl. Despite the smaller volume V₂ at the base of each cell 600, the relatively small depth of this smaller volume and the larger volume V₁ on top of that allow the necessary amount of analyte for photometric analysis to be placed into the cell without that analyte extending too far up the cell. However, for TPX analysis, a relatively small volume of analyte is necessary, typically between 40 and 50 μl. The small volume V₂ at the base of each cell accommodates this relatively small volume. Prior art cuvettes, having a rectangular section, tend to have a minimum fill volume which is inappropriate to the small volumes of analyte necessary for TPX analysis. For example, the cuvette described in the above-referenced U.S. Pat. No. 4,690,900 has a minimum fill volume of 100 μl. Thus, by employing a smaller volume at the base of each cell, the amount of sample and, importantly, the volume of reagent necessary where TPX analysis is to be carried out is reduced, by 50% or more.

An associated benefit with the particular shape in FIG. 3a and FIG. 3b is an improved ability to mix sample and reagent within the cell. Although stirring may be done in different directions, either magnetically, mechanically or otherwise, the tapered base to each cell allows mechanical stirring, using a propellor, for example, to be achieved more effectively. This is particularly the case when very small volumes need to be stirred.

In addition, the use of a smaller volume at the base of each cell in the cuvette optimizes sample storage. In any typical alternated analyzer, there will be a minimum gap between the needle end and the base of the sample/reagent storage (a minimum gap must be specified to avoid damage to the needle should it come into contact with the base of the sample/reagent storage). By reducing the sectional area of the base of each cell, for a given minimum gap between the needle that uptakes the fluid and the base of the cell, the “dead volume” is minimized.

The reduced sectional area at the base of the cuvette cell also improves the reliability of the surface detection. The surface detector of the needle is not able to distinguish between the surface of the sample and an empty but wet cell. When the cell is very close to empty, the tolerance of the needle height adjustment may cause a sample uptake from a volume which is too small to fulfil the desired sample volume. This will lead to a false low result. By reducing the sectional area of the volume V₂, the same sample volume will result in a higher sample “pillar” in the cell and thus in turn allow a higher height adjustment tolerance, resulting in a more reliable uptake from small sample volumes.

Finally, the smaller volume V₂ also allows hemolysis/icterus/lipemia (HIL) interferences to be measured more readily since such HIL detection again requires low sample volumes.

The use of the air gap 620 permits improved thermal isolation between analytes in the smaller volume V₂ of adjacent cells. Minimizing heat transfer between adjacent cells improves temperature stability.

For photometric analysis, if heat transfer is to be minimized, then alternate cells (600a, 600c, 600e, 600g, 600i and 600k, for example) can be employed.

The front wall 630 (FIG. 3b) of the cuvette 60 may be provided with marks, spots, lines or the like. As seen in FIG. 3a, the lower volume V₂ employs a first circular mark 650 and a larger diameter circular mark 660 is employed above that within the larger volume V₁. These circular marks 650, 660 permit automatic adjustment of the measuring position of the cuvette 60 and in particular a specific cell 600 of that cuvette, relative to the incident light used for analysis. For example, light reflected from the front face 630 of the cuvette 60 may be detected and analyzed by the controller 170 (FIG. 1) to control cuvette driving robotics (not shown) under feedback. The lower and upper circular marks 650, 660 can be used to move the cuvette 60 to address differing measuring requirements in the different volumes V₂ and V₁. A hook 670, similar to that employed in U.S. Pat. No. 4,690,900, can be provided on a side of the cuvette 60 for engagement with cuvette driving robotics to allow movement of that cuvette relative to a light source during analysis.

Although circular marks of differing diameters are shown in the arrangement of FIG. 3a, it will be understood that these are merely exemplary and that any form of mark, spot, line or other index could be used to allow control of the location of IS the cuvette 60. Likewise, whilst such an index has been shown on the front face of the cuvette in FIG. 3a, it or they could of course be formed on a rear wall 640.

In some applications, it may be desirable to illuminate the analyte from underneath as viewed in FIG. 3a and an optical window 680 is provided in the base of each cell 600 for that purpose.

Whilst a specific embodiment of a cuvette has been described, it will be appreciated by those skilled in the art that various modifications are possible. For example, rather than employing a lower volume V₂ in which its sectional area is entirely non-constant, and an upper volume V₁ whose sectional area is entirely constant, different combinations could instead be employed. In one embodiment, a first, lower volume of constant section may be provided, linked to a second, upper volume also of constant sectional area via an intermediate volume which tapers from the smaller to the larger sectional area. Alternatively, a cell may have an entirely non-constant cross-sectional area from its base to the opening at the top—that is, each cell may be tapered from top to bottom, either uniformly or otherwise. In that case, the upper and lower volumes are divided from each other arbitrarily since there is no point of discontinuity in the shape of the dividing walls but upper and lower volumes may nevertheless be defined and the claims appended hereto are to be construed as such.

Likewise, the arrangement of the analyzer of FIG. 1 is merely exemplary. For example, although the TPX and photometric analysis arrangements are shown adjacent one another in that Figure, the skilled person will understand that this is not essential. It may be beneficial, in terms of efficiency of parallel processing of samples, to have the photometric and TPX analysis arrangements 150, 160 adjacent to one another. However, there may be benefits in having these arrangements, for example, on opposing sides of the analyzer 10.

1. An automated sample analyzer comprising:
a sample repository arranged to hold a plurality of samples to be analyzed;
a reagent repository arranged to hold a plurality of reagents to be mixed with the samples;
an analyte preparation station arranged to receive a quantity of sample from the sample repository, to receive a quantity of at least one reagent from the reagent repository, and to mix these together to form an analyte;
a photometric analysis assembly arranged to carry out a photometric analysis of a material;
a twin photon excitation (TPX) assembly arranged to carry out twin photon excitation (TPX) analysis of a material; and
a controller to divert the analyte from the analyte preparation station to the photometric analysis assembly or the TPX analysis assembly in dependence upon the desired type of analysis of the analyte.

2. The automated sample analyzer of claim 1, further comprising a cuvette repository arranged to hold a plurality of cuvettes, the analyte preparation station being further arranged to receive a cuvette from the cuvette repository and to dispense that cuvette containing the analyte for onward passage to the one of the photometric or TPX analysis assemblies.

3. The automatic sample analyzer of claim 2, wherein the analyte preparation station is arranged to dispense both the received quantity of the sample and the received quantity of reagent into the cuvette, to mix these in the cuvette, and then to dispense that cuvette with the analyte therein for the said onward passage.

4. The automatic sample analyzer of any preceding claim, wherein the analyte preparation station is arranged to hold a mixed sample and reagent, once received, for a predetermined time so as to permit incubation thereof prior to dispensing the analyte for analysis.

5. The automatic sample analyzer of any preceding claim, wherein the controller is further arranged to control the quantities of sample and/or reagent which are obtained from the sample and the reagent repositories respectively, in dependence upon the desired type of analysis of the resultant analyte.

6. A cuvette for use in an automated sample analyzer, the cuvette comprising a chamber having chamber walls which define a chamber opening and first and second chamber volumes, wherein the first volume is proximal the opening, the second volume is remote from the opening, and wherein the first volume has a transverse sectional area larger than the transverse sectional area of the second volume.

7. The cuvette of claim 6, wherein at least one of the first and second volumes has a constant transverse sectional area.

8. The cuvette of claim 6 or claim 7, wherein at least one of the first and second volumes has a transverse sectional area that reduces in a direction moving away from the chamber opening.

9. The cuvette of claim 6, wherein the first and second volumes are contiguous and wherein the transverse sectional areas of each reduce in a direction away from the chamber opening so that the chamber has a tapered longitudinal section.

10. The cuvette of any of claims 6 to 9, wherein at least one of the chamber walls is translucent.

11. The cuvette of any of claims 6 to 10, wherein at least one of the chamber walls includes an indicator to allow alignment of a sample measurement device with a predetermined part of that chamber wall.

12. The cuvette of any of claims 6 to 11, wherein the cuvette comprises a second chamber having second chamber walls, the second chamber being adjacent to the first chamber, and wherein the first and second chambers are spaced from one another by a buffer region.

13. The cuvette of claim 12, wherein the buffer region is defined between opposing chamber walls of the first and second chambers.

14. The cuvette of claim 13, wherein the buffer region includes an air gap between the said opposing chamber walls.

15. The cuvette of claim 13 or claim 14, wherein the second chamber walls define first and second chamber volumes within that second chamber, and wherein the width of the buffer region between corresponding first volumes of the first and second chambers is less than the width of the buffer region between corresponding second volumes of the first and second chambers.

16. The cuvette of any of claims 6 to 15, wherein the walls define a base to the or each chamber, the walls being tapered or curved in a direction longitudinally of the chamber opening in the direction of the base.

17. In combination, the automated sample analyzer of any of claims 1 to 5 and the cuvette of any of claims 6 to 16, in which is contained the said analyte.

18. In combination, the automated sample analyzer of any of claims 1 to 5 and the cuvette of claim 11, the controller of the automated sample analyzer being configured to divert the cuvette, containing the analyte, to the photometric or TPX analysis assembly, in dependence upon the desired type of analysis of the analyte, the controller being further configured to control the alignment of the said cuvette relative to the respective photometric or TPX analysis assembly in accordance with the said indicator on the at least one chamber wall of the cuvette.

19. A method of automated sample analysis comprising the steps of:
(a) storing at least one sample to be analyzed;
(b) storing, separately, at least one reagent to be mixed with the sample(s);
(c) receiving a quantity of a sample and a quantity of the, or at least one reagent;
(d) mixing the quantities of sample and reagent(s) to form an analyte; and
(e) directing the analyte to a selected one of a photometric analysis assembly and a twin photon excitation (TPX) analysis assembly in dependence upon the desired type of analysis of the analyte.

20. The method of claim 19, further comprising incubating at least one of the quantity of sample, the quantity of reagent(s) and a mixture of these in cuvette for a predetermined time period.

21. The method of claim 19 or claim 20, further comprising controlling the quantities of sample and/or reagent(s) that are received in dependence upon which of the photometric and TPX analysis is to be carried out.
